(19) United States
(12) Patent Application Publication
(10) Pub. No.: US 2025/0146045 A1
(43) Pub. Date: May 8, 2025

(54) METHODS FOR DETERMINATION OF BACTERIA IN SAMPLES

(71) Applicant: IDEXX Laboratories, Inc., Westrbook, ME (US)

(72) Inventors: Jeremy Hammond, Standish, ME (US); JuiMing Lin, Falmouth, ME (US); Julia Chase, Westbrook, ME (US); Seana Gray, Kennebunk, ME (US); Sarah Roger, Barrington, NH (US)

(21) Appl. No.: 18/941,160

(22) Filed: Nov. 8, 2024

Related U.S. Application Data
(63) Continuation of application No. 63/674,376, filed on Jul. 23, 2024.
(60) Provisional application No. 63/547,700, filed on Nov. 8, 2023.

(51) Int. Cl. C12Q 1/06; G01N 21/64

(52) U.S. Cl. CPC C12Q 1/06; G01N 21/6428; G01N 21/6458; G01N 2021/6439; G01N 2201/127; G01N 2333/195; G01N 2333/24; G01N 2333/245

(57) ABSTRACT

An apparatus and method for detecting the type, presence or amount of bacteria in a sample. The apparatus and method involve staining a liquid sample with a fluorescent stain corresponding to bacteria and imaging one or more portions of the sample at a selected set of sample Z-axis heights, X-Y coordinates, or both, within the sample after a predetermined time to allow for non-bacterial components in the sample to settle out of solution. A calibration method for correlating the concentration or amount of bacteria in biological samples by different analysis methods including glass slide methods, agar plate colony culture methods and high precision methods.

### RELATED APPLICATIONS

This application claims priority to U.S. Provisional Application Nos. 63/547,700 and 63/674,376 filed Nov. 8, 2023 and Jul. 23, 2024 respectively, which are incorporated herein by reference in their entirety.

### FIELD

The disclosure is directed to the determination of presence, type and/or quantity of bacteria in various types of biological, industrial and environmental samples.

### BACKGROUND

Methods of identifying bacteria in various types of biological, industrial and environmental samples suffer from technical challenges. Bacteria is difficult to identify using microscopy because the cells are small, lack morphologic features, and are hard to differentiate from non-bacteria debris. Light microscopy ultimately only allows bacteria to be visualized as they appear in natural light. When evaluating bacteria in a bulk fluid for some biological samples using a regular brightfield microscopy approach, it becomes a technical difficulty to discriminate bacteria from other similar objects such as cell debris and dust since their size is similar in a range of 0.5-2.5 μm and they have minimal morphologic variation.

While quantifying an amount of bacteria in a sample is possible by several methods, there is no standard procedure to compare one method to the other. Glass slide interpretations of bacteria samples have been used for decades. While there remain several uncontrolled variations in the methods, there have been rules of thumb for defining how to interpret the relative quantities of bacteria on a slide. Generally, semi-quantitative buckets are defined to size the population. However, there is little standardization, and the method is not well suited to having high precision. While high precision methods are available, the marketplace will benefit from a method that correlate such methods to glass slide interpretation.

### SUMMARY

The disclosure provides a simple and accurate method to quickly identify presence, type and/or quantity of bacteria in samples.

One aspect of the disclosure is directed to method for detecting a type, presence or amount of bacteria in a liquid. The method includes staining a liquid sample with a fluorescent stain for bacteria, introducing the stained liquid sample into a sample container having a Z-axis height that allows for settling of components of the liquid sample; allowing the components in the liquid sample to settle for a predetermined amount of time; imaging a Z-axis field of view (FOV) comprising an increment of the Z-axis height in the liquid sample at a location comprising an X-axis and Y-axis coordinate (X-Y coordinate); and determining the presence or absence of bacteria within the FOV and correlating the type, presence or amount of bacteria in the FOV to the type, presence or amount of bacteria in the liquid sample.

In various aspects of the disclosure, an image of the FOV is obtained with a confocal microscope or an epifluorescence (EPI) microscope and/or the imaging includes obtaining a plurality of FOVs at the X-Y coordinate and correlating the type, presence or amount of bacteria in the plurality of FOVs to the type, presence or amount of bacteria in the liquid sample. In one aspect, the imaging obtaining a plurality of FOVs at the X-Y coordinate and correlating the type, presence or amount of bacteria in the plurality of FOVs to the type, presence or amount of bacteria in the liquid sample.

Further aspects of the disclosure include the container having a Z-axis height between about 0.1 to 1 mm and/or a depth of field of the increment of the Z-axis height is about 1 μm to 10 μm.

In another aspect of the method of the disclosure, determining of bacteria within a FOV includes comparing an image of the FOV to a predetermined image indicative of the presence or type of bacteria in the FOV. In various aspects, the imaging includes determining a fluorescence spectra of stained bacteria in the FOV and/or the imaging includes determining a fluorescence spectra of stained bacteria in the FOV and determining a strain of the bacteria in the FOV based upon the fluorescence spectra. In addition, the method of the disclosure may include obtaining a bright-field image of the FOV.

In each of the foregoing aspects of the disclosure may include processing a native human, animal, environmental or industrial sample to obtain the liquid sample by mixing the native sample with a reagent comprising a surfactant and a buffer. The native sample may be urine or a body cavity fluid or may be a solid, highly viscous, or semi-solid sample, for example ear cerumen, peripheral blood, solid tumor, or fine needle aspirate.

In yet another aspect, the disclosure is directed to a type, presence or amount of bacteria in biological sample with a method including the following; mixing the biological sample with a suspension liquid to provide a liquid sample and staining the sample with a fluorescent stain for bacteria, introducing the stained liquid sample into a sample container having Z-axis height that allows for settling of components of the liquid sample; allowing the components in the liquid sample to settle for a predetermined amount of time, imaging a Z-axis field of view (FOV) comprising an increment of the Z-axis height in the liquid sample at location comprising an X-axis and Y-axis coordinate (X-Y coordinate); and determining the presence or absence bacteria within the FOV and correlating the type, presence or amount of bacteria in the FOV to the type, presence or amount of bacteria in the liquid sample.

Still further, each of the aspects of the disclosure can include analyzing an image with a computing device coupled to or in communication with a camera that images a FOV and executes instructions stored in a memory of the computing device.

In one aspect, the disclosure is directed to a method for calibrating a bacteria detection method. The method includes
(a) counting bacteria from a biological sample using a glass slide method to obtain a reference count,
(b) culturing bacteria from a portion of the biological sample on a first plate to provide a first bacteria culture;
(c) counting the bacteria on the first plate;
(d) collecting a first plate sample from the plate having a first predetermined quantity of bacteria;
(e) analyzing the first plate sample in a high precision analysis method for determining an amount of bacteria in the first plate sample;
(f) correlating the amount of bacteria determined in step (e) to an amount of bacteria in the plate sample determined in step (c), and
(g) comparing the amount of bacteria in the first plate sample to the amount of bacteria in the reference count.

In this aspect, the high precision analysis method may include the following steps:
(i) combining at least a portion of the first plate sample with a fluorescent stain for bacteria to provide an analysis sample,
(ii) introducing the analysis sample into a sample container having a Z-axis height that allows for settling of components of the analysis sample;
(iii) allowing components in the analysis sample to settle for a predetermined amount of time;
(iv) imaging a Z-axis field of view (FOV) comprising an increment of the Z-axis height in the liquid sample at a location comprising plurality of X-axis and Y-axis coordinates (X-Y coordinates); and
(v) determining the amount of bacteria within the FOV.

This aspect may further include repeating steps (d) through (g) with a second plate sample comprising a second predetermined quantity of bacteria collected form the first plate or a second plate comprising a second bacteria culture comprising bacteria from the biological sample cultured on a second plate. In various embodiments, the first plate sample and second plate sample are diluted prior to step (e). In addition, the first and the second predetermined quantities of bacteria are determined by measuring the optical density of the first and the second diluted plate samples.

In further aspects, the methods of the disclosure include collecting at least a portion of the first bacteria culture with a swab or a loop, and optionally further including collecting at least a portion of the second bacteria culture with a swab or a loop. The bacteria on the plate may be counted by counting colony forming units on the plate.

Still further, the methods of the disclosure may include preparing a transfer function to correlate the amount of the bacteria on the plate to the amount of bacteria in the reference count.

In another aspect of the methods of the disclosure, the amount of the bacteria measured on the glass slide in step (a) is identified within one of a plurality of defined ranges of quantities of bacteria. The defined ranges may be correlated to clinical decision points associated with bacteria in a biological sample and resulting in a diagnosis of infection.

In another aspect of the methods of the disclosure, the quantity of bacteria measured in step (e) is identified at within one of a plurality of defined ranges of quantities of bacteria.

In another aspect of the methods of the disclosure, the imaging comprises obtaining an image of the FOV with a confocal microscope, brightfield microscope, or an epifluorescence (EPI) microscope.

In another aspect of the methods of the disclosure, the imaging comprises obtaining images of a plurality of FOVs at the X-Y coordinate and correlating the type, presence or amount of bacteria in the plurality of FOVs to the type, presence or amount of bacteria in the liquid sample. The Z-axis height may be between about 0.1 to 1 mm. Also, a depth of field of the increment of the Z-axis height may be about 1 μm to 10 μm.

In another aspect of the methods of the disclosure, the amount of bacteria within the FOV comprises comparing an image of the FOV to a predetermined image indicative of the presence or type of bacteria in the FOV.

In another aspect of the methods of the disclosure, the imaging includes determining a fluorescence spectra of stained bacteria in the FOV.

In another aspect of the methods of the disclosure, the native sample is a solid, highly viscous, or semi-solid sample.

In another aspect of the methods of the disclosure, the sample is ear cerumen, peripheral blood, a solid tumor, or fine needle aspirate. Also, the sample may be urine or a body cavity fluid.

In another aspect of the methods of the disclosure, the methods include obtaining a bright-field image of the FOV.

In another aspect of the methods of the disclosure, the determining includes analyzing an image with a computing device coupled to or in communication with a camera that images a FOV and executes instructions stored in a memory of the computing device.

### BRIEF DESCRIPTION OF FIGURES

The accompanying drawings, which are included to provide a further understanding of the disclosure, are incorporated in and constitute a part of this specification, illustrate embodiments of the disclosure, and together with the detailed description serve to explain the principles of the disclosure. No attempt is made to show structural details of the disclosure in more detail than may be necessary for a fundamental understanding of the disclosure and various ways in which it may be practiced.

FIG. 1 shows a schematic representation of a device for high precision analysis of samples according to an aspect of the disclosure.

FIG. 2 shows representative comparison of trained algorithm for counting bacteria using a high precision method against reference plate counts for algorithm classifier training according to an aspect of the disclosure.

FIGS. 3A and 3B show a representative transfer function plot (3A) of a high-precision bacteria counting method compared to traditional glass reference method and zoomed in on the clinically relevant decision area (3B).

FIG. 4 shows a confocal 3-D rendering image for fluorescence-stained cocci obtained according to a method of the disclosure.

FIG. 5 shows a confocal 3-D rendering image for fluorescence-stained bacteria rods according to a method of the disclosure.

FIG. 6 shows 3-D rendering images for bacteria and dust dispersion in a liquid sample after a settling time of 20 minutes.

FIG. 7 shows a transfer function comparing the bacteria determined by a glass slide method and a high precision method.

FIG. 8 shows a representation of a transfer function determined according to a method of the disclosure.

### DESCRIPTION

In various aspects, the disclosure is directed to calibration method for a system and methods for determining type, presence and/or quantity of bacteria in samples, such as biological samples collected from humans and animals, environmental samples and industrial samples. The calibration method includes the standardization between various bacteria counting methods, for example between a quantity of bacteria measured using a glass slide method and a quantity of bacteria measured using a high precision method. Such high precision methods can include a system with an inverted microscope that identifies fluorescently-labeled bacteria in the samples that are processed to render the bacteria and other sample components suspended in a processed liquid sample.

In various aspects, the calibration method of the disclosure includes culturing bacteria on agar plates, counting the bacteria on the plate (e.g., by counting colony forming units on the plate), and transferring the bacteria to a swab or other collection device to simulate gathering bacteria from a biological sample, such as ear cerumen. The method includes reproducibly loading a cotton swab or other collection device with an amount of bacteria and bacteria load that can be controllably varied on the swabs. By reproducibly collecting various known amounts of bacteria from agar plates with a swab or other collection device, a transfer function between a reference method (e.g., glass slide method) and a high precision method can be developed. The transfer function allows a correlation between an amount identified from the reference method (a reference count) to an amount identified by a high precision method.

In an aspect of the disclosure, the collection of a culture for use in the reference method mimics the collection of bacteria from a natural sample (e.g., collection of ear cerumen from an animal). Therefore, the transfer function can also be used to correlate the amount of bacteria identified in a natural sample by a reference method to the amount of bacteria in the sample identified by a high precision method. While a high precision method is intended to provide an accurate result, the result may not be particularly helpful to a user that has traditionally relied on clinical thresholds from the reference method. Therefore, an accurate correlation between the reference method and a high precision method can assist the user in deciding a next step, for instance a decision regarding treatment of a bacterial infection.

In the case of glass slides, quantitation of bacterial counts suffers from inaccuracy as a result, for example, of lack of homogeneity of a sample, the presence of non-bacterial cells and other matter, and the training and variability between technicians. Therefore, laboratory bacteria results are often reported as falling within a range of quantities or “buckets” that can be useful surrogates (e.g., normal, moderate, marked or abnormal, gray) from a quantitative result. The ranges or buckets are indicative of the amount of bacteria identified in a reference method, for example a glass slide method as shown in Table 1.

TABLE 1

| Animal Cerumen | | | |
|---|---|---|---|
| Sample | Normal* | Gray Zone* | Abnormal |
| Dog | 0 or ≤5 | 6-24 | ≥25 |
| Cat | 0.3 or ≤4 | 5-14 | ≥15 |

*Median number or average number organisms per 40x high power focus (hpf). See Ginel, et al., A semiquantitative cytological evaluation of normal and pathological samples from the external ear canal of dogs and cats, Vet Dermatol. 2002 June; 13(3): 151-6.

Thresholds are critical for separating the quantitative data into the desired buckets, which can mimic current information available to, for example, veterinarians that have traditionally relied on the buckets in animal health care decision. However, there is little existing literature documenting clinical cutoffs on glass slide preparations, and those that exist were generated from small data sets (e.g. tens of animals).

In various embodiments, the swab or other collection device is a natural or synthetic fiber swab, a collection loop, or similar device that can be used to reproducibly collect an amount of bacteria from an agar plate. Reagents necessary for processing and analysis of the sample collected with the swab or device, either from an agar plate or a natural source, can be the same, regardless of whether the analysis method involves a glass slide or high precision method.

In one aspect of the disclosure, the authenticity of a sample collected from an agar plate can be evaluated by analysis of the contents of the bacteria-spiked swab and a natural swab containing other natural ear elements (e.g., mites, dust, epithelial cells, cell debris, wax, cells (e.g., white blood cells, red blood cells) and yeast) or even spiked with, for example, white blood cells and/or yeast. Collected data from these samples can be used in an algorithm that can be optimized for reporting the metric for bacteria load in a high precision method. Bacteria loads on the glass slide from repeated agar plate swabs and the amount of bacteria on the swab can be modified by the size of the colony on the agar plate and the force and duration of contact between the swab and the colony.

In one aspect, the disclosures is directed to a transfer function that correlates the results from a reference method to a high precision method without having to explicitly define the factors and their impacts on the results, such as dilution, sample volume, etc. In an aspect, a transfer function is generated from a large number of natural ear samples with varying amounts of bacteria present and analyzing each sample using both the reference method and a high precision method. For the reference method, the best approach is to count bacteria present. A correlation plot of the reference counts against the high precision method outputs provides the transfer functions by creating a regression analysis. In aspects, the transfer function is not linear, and a variety of logistical regression approaches can be used to determine the transfer function.

In an example glass slide reference method, a cotton swab is rubbed in the ear of an animal and then rubbed on a glass slide. The slide is then stained with a methanol-based stain, commonly with a Gram Stain (e.g., Diff Quick, Dermcare-Vet Pty Ltd.) The slide is then reviewed under a microscopic objective (e.g., 40×, 100×) and bacteria are counted and classified within one of several ranges or buckets of bacteria counts as described above. In some aspects, the bucket thresholds change on glass with magnification and can be scaled by the square of the ratio of magnifications. As an example, if a threshold at 40× is 5, then the threshold at 100× would be reduced by a factor of 1/(2.52) and would result in an average count of 0.8). Other glass slide reference methods are also contemplated.

A high precision method of determining bacteria counts can use the same swab process as the reference method. Following sample collection, the swab is introduced into a diluent for preparation with fluorescent stains. The prepared sample is then dispensed into a cartridge for analysis. As further described herein, white blood cells and similar cells will settle to the bottom of the cartridge at a rate of about 1-micron per second. Bacteria will settle at a much slower rate and will therefore not be fully settled at the cartridge bottom when scanning will occur. Images can be captured higher in the cartridge above the depth of field where the large, settled objects show up.

### Transfer Function

In one aspect, the disclosure is directed to a transfer function that correlates the results from a reference method to a high precision method without having to explicitly define the factors and their impacts on the results, such as dilution, sample volume, etc. In various aspects, a transfer function is generated from a large number of samples collected from agar plates and/or from natural ear samples with varying amounts of bacteria present and analyzing each sample using both the reference method and a high precision method. A correlation plot of the reference method counts against the high precision method outputs provides the transfer function by creating a regression analysis. In aspects, the transfer function is not linear, and a variety of logistical regression approaches can be used to determine the transfer function. Use of samples having known amounts of bacteria from agar plates provides a confirmation of the process for natural samples.

Given the relatively large natural variation found from the reference method based on variation in creating the glass slide, efficiency of transferring bacteria from the swab to the slide, and variation in evaluating different aspects of the slide, the correlation between the reference method and higher-precision method will generally have a lower than desired correlation coefficient. The transfer function is intended to use various approaches to identify the central trend of the data and provide a relationship between the two methods with enough power to generalize the function for scaling the clinical thresholds. This is counter to many method comparisons where high correlation coefficients are expected to demonstrate equality of the methods since the higher-precision method may out-perform the reference method. The transfer function provides a means to extract the clinically relevant thresholds outside of extensive clinical trials.

### High Precision Analysis of Samples

One aspect of the disclosure allows for the evaluation of bacteria on a fluorescence microscope in a general biology or clinical laboratory without access to a microbiology level test like polymerase chain reaction (PCR) or other molecular diagnostic techniques. In addition, the approach includes universal fluorescence staining of bacteria that provides a time-saving method compared to methods involving bacteria enzymatic reaction. Universal fluorescence staining is generally applicable to most bacteria types. Accordingly, the methods of the disclosure and associated kits and components represent a cost-saving and labor-saving way to evaluate bacteria compared to known approaches.

Bacteria can be divided into two major groups, gram-positive and gram-negative, and there are three basic bacteria shapes: spheres or ball-shaped (cocci bacteria), rod-shaped bacteria (bacilli), and spirals or helixes (spirochetes). In one aspect, the system and method of the disclosure allow for determination of the type, presence or amount of bacteria in the sample. Determining a type of bacteria includes, for example, differentiating whether the sample includes cocci, bacilli or both.

The system detects and quantifies bacteria in collected samples when the sample components are suspended in a liquid diluent. Samples can include any type of biological, industrial and environmental samples to the extent that the sample is liquid and/or the sample components can be suspended in a liquid medium. Typical biological samples include blood, urine, feces, saliva, body cavity fluids (cerebral-spinal fluid, synovial fluid (joints), thoracic fluid, pleural fluid, abdominal fluid) tumor samples, ear cerumen, skin swabs/scrapes and fine-needle aspirates that can be suspended in a liquid medium and analyzed for bacteria according to the methods of the disclosure.

As used herein, the term “sample” may refer to either an unprocessed (“raw” or “native”) sample in any form (fresh, frozen, etc), or “sample” may refer to a native sample that has been processed for analysis according to the disclosure (e.g., a processed sample), the difference between the samples is apparent from the context of the disclosure. In some aspects of the disclosure further described herein, the type, presence or amount of bacteria in a processed sample analyzed according to the disclosure is correlated to the type, presence or amount of the bacteria in a native, unprocessed sample.

### Sample Processing and Staining

Biological samples from humans, animals, industry and the environment can be collected using known techniques. Solid, semi-solid, or highly viscous samples, such as ear cerumen, peripheral blood, and fine needle aspirates, provide unique challenges when preparing the sample to be analyzed on, for example, glass slides. For instance, each has varying viscosity, component concentration and complicating interferences (e.g., lipids, wax, clumped cells, tissue). These factors, as well as technician skill, lead to frequent slide preparation difficulties and inconsistencies that can hinder efficacy of interpreting these samples via manual microscopy. The most common problems with manual slide preparation are the lack of a true monolayer, mechanical damage to cells, lack of homogeneity, and poor or inconsistent staining. Additionally, in typical manual glass slide microscopy analysis, small component assessment can be challenging amongst larger components that are in the same coincident space. Ear cerumen (ear wax), in particular, is a difficult sample because it is inconsistent (very hard to very soft), contains multiple constituents and contaminants, and is typically available in a small amount. Avoiding sample-to-sample variation such that sample contents can be consistently analyzed proves challenging for a laboratory technician.

In order to address these and other sample analysis challenges, the present disclosure includes the use of stabilizing diluents, buffers and surfactants for preserving native cell morphology and assuring homogenous distribution of non-dissolving sample components within a processed sample that is analyzed according to the disclosure. The sample, once processed as described herein to be in liquid form, can be added to a specified volumetric microscopy container that provides an alternative to typical manual microscopy preparation challenges and leads to preparation standardization and superior accuracy.

As used herein, “non-dissolving sample components,” or simply “sample components” or “components,” include sample substituents that have sufficient mass to cause the components to settle over a predetermined amount of time. Example sample components include, for example, cells (e.g., bacteria, blood cells), organisms (e.g., mites), and debris (e.g., dust, pollen). The microscopic analysis techniques of the disclosure can differentiate bacteria in a processed sample from other components of the sample. Dissolved sample substituents that do not settle are not sample components.

Native samples containing bacteria can be obtained, for example, from bacteria culture (e.g., agar plates and/or liquid media), bacteria infected fluid, and tissue swabs or collection devices. The native samples can be suspended in an isotonic buffer solution (suspension liquid) with neutral pH to provide a processed sample containing a homogeneous mixture of non-dissolvable sample components. To facilitate the suspension of the non-dissolving sample components into a homogeneous liquid sample, the liquid samples may be vortexed for a few seconds to break apart aggregated bacteria or extract bacteria from the sample collection devices. Alternatively or additionally, the liquid samples may be incubated in a 37° C. water bath for a short period of time (e.g., one minute).

Surfactants in the bacteria suspension liquid can facilitate the suspension process, compromise cell wall, and facilitate the stain uptake. Example surfactants can include cationic, anionic, non-ionic, and zwitterionic types of chemicals. Cationic surfactant are substances that bear positive charges such as docosyltrimethylammonium chloride; anionic surfactant are substances that bear negative charges such as Sodium dodecyl sulfate (SDS); nonionic surfactants contain no charge such as Tween 20™ (polysorbate 20); zwitterionic surfactants belong to the class of surfactants that is composed of both positive and negative charges such as Cocamidopropyl betaine. Surfactant concentration in a liquid sample can range from about 0.001% to about 0.5%. For example, about 0.001%, 0.005%, 0.01%, 0.05%, 0.1%, 0.2%, 0.3%, 0.4% and 0.5%.

The bacteria suspension liquid may include pH buffering agents and salts (sodium chloride, Tris-HCl (Tris (hydroxymethyl) aminomethane hydrochloride)), Tris-base (Tris (hydroxymethyl) aminomethane)), and EDTA (Ethylenediaminetetraacetic acid)). Examples of the bacteria suspension liquid may include these buffering agents, but not limited to, vary from 10 mM to 200 mM for each buffering component.

An example suspension liquid for processing native samples can include sodium chloride, Tris-HCL, EDTA, and Tris-base. In embodiments, example concentrations of these ingredients may include the following: about 6-10 g/L of sodium chloride, 1.4-2.0 g/L of Tris-HC1, 0.6-1.0 g/L of EDTA, and 2.0 to 1.76 g/L of Tris-base. An example combination of these ingredients includes the following: about 7.9 g/L of sodium chloride, 1.65 g/L of Tris-HCl, 0.8 g/L of EDTA, and 2.35 g/L of Tris-base.

Sample bacteria can be fluorescently-labeled by methods known in the art. For example, nucleic acid staining dyes and cell surface markers tagging by antibodies (e.g., anti-E. coli antibody, anti-pseudomonas aeruginosa antibody, and the like) or aptamers (e.g., single-stranded DNA or RNA) conjugated with fluorescence probes. Bacteria cell wall permeable fluorescent nucleic acid staining dyes can be used, for example Acridine Orange or SYTO-13™ (both Thermo Fischer), or thiazole orange, along with other intracellular nucleic acid staining dyes.

An important aspect of nucleic acid staining efficiency in bacteria includes bacteria staining variance by different strains due the structural differences of cell walls when uptaking the staining dyes. Some universal nucleic acid stains do not achieve an optimal staining due to low fluorescent dye uptake. Therefore, to increase the staining efficiency, the bacteria samples can be incubated at elevated temperatures (e.g., water bath or an oven at 25-37° C., or a dedicated heating system). Alternatively or additionally, a low concentration of one or more surfactants can be added to the bacteria samples. The surfactants compromise the cell wall, which allows fluorescent dyes to diffuse rapidly inside the bacteria while maintaining the shape of the cell wall. Both thermal and surfactant treatments can be combined to achieve an optimal staining condition.

Nucleic acid stains for bacteria cover a wide range of different bacteria strains and can provide a sufficient signal-to-noise ratio (SNR) to evaluate bacteria in an assay designed to count bacteria. As one example, SYTO-13™ stain is a universal fluorescent stain for bacteria that can provide a signal to noise ratio sufficient for bacteria identification. Noise includes background fluorescence (autofluorescence) and any non-bacteria elements that may be fluorescing. Signal includes the magnitude of fluorescent intensity from bacteria. Sufficient SNR can occur when the bacteria signal is clearly separated from the noise signal. An example of a sufficient SNR is 2:1 of signal to noise, and higher values can enhance performance. In one example, bacteria presence and absence in the sample is determined when a threshold of fluorescent signal in the liquid sample solution is above the background noise floor. In another example, an E coli. bacteria sample is introduced to a resuspension solution containing a cell permeable nucleic acid dye (Acridine Orange), 0.01% Tween 20™ (polysorbate 20), and 100 mM phosphate pH 7.5. The suspension solution can be incubated in a 37 C water bath to increase dye uptake.

### Sample Container

As described further herein, an epifluorescence (EPI) or confocal microscope can be set up to optically scan a sample container, such as a custom cartridge, chamber, or slide (e.g., cell counter slide) made up with glass or plastics and, in some embodiments, without auto fluorescence.

Once the suspension liquid is mixed with the sample to provide a liquid sample, the mixture may be loaded into a sample container. In some aspects, microbubbles in the container are avoided and the container is completely filled. No seal over the loading port or vent is necessary if a sample is analyzed in a short amount of time (e.g., minutes) such that sample evaporation does not affect the analysis. A mineral oil may be used to seal a sample loading port and vent holes in the container to prevent sample evaporation (since the objects may move in any X-Y-Z motion during the image acquisition when water evaporation occurs) depending on the amount of time need for analysis of container contents. Parafilm and other materials that effectively inhibit evaporation without interacting with or contaminating the sample may be used. Container/sample scanning run times of less than ten minutes are generally not affected by evaporation, but the mineral oil or similar seal may be considered for longer runs. Unless otherwise apparent from the context of the disclosure, the volume of the sample container and the sample are congruent such that the container is essentially completely full.

The sample container should have sufficient depth, referred to herein as the Z-axis depth, that will allow the non-dissolving components (e.g., bacteria) in the processed sample liquid to separate and ultimately settle at the base of the container. Because bacteria are smaller than most other sample components, they will remain suspended in the liquid sample longer than most other components. Non-bacteria components usually settle out of solution within minutes such that the analysis of the liquid sample can be conducted in less than 10 minutes.

The depth of the container should allow for settling of non-bacteria components while providing sufficient sample height in order for the settling characteristics of the bacteria in solution to be analyzed. Containers having a Z-axis height of about 50 to about 500 micrometers (micron or μm) provide sufficient depth that allow for settling of non-bacteria components within a time period desired for analysis in a clinical laboratory or point-of-care setting. For example, container heights 50 μm, 100 μm, 150 μm, 200 μm, 250 μm, 300 μm, 350 μm, 400 μm, 450 μm and 500 μm may be used. In some embodiments, the sample container height is between 100 μm and 300 μm. In general, shorter depths can have more non-focused fluorescence light generated in a bulk fluid that may be captured in a camera attached to the microscope.

Total volume of the sample within a container may range from 0.5 μl to about 100 μl, for example about 5 μl, 50 μl, 100 μl, 200 μl, 300 μl, 400 μl, 500 μl, 600 μl, 700 μl, 800 μl, 900 μl, or 1000 μL.

Sample container height may vary depending on the type of sample that is analyzed. An example commercially available container includes an InCyto C-Chip disposable slide (iNCYTO) or an Sight OLO cartridge (Sight Diagnostics), which include an enclosed volume that can be filled and then analyzed with a microscope as disclosed herein. Other configurations can address system constraints and methods of heating as described herein.

As one example, a sample container is an IDEXX in Vue Dx™ sample cartridge or a cartridge described in U.S. provisional application Ser. No. 63/615,571, which is incorporated by reference herein.

### Apparatus for High Precision Bacteria Counting

In various aspects of the disclosure, epifluorescence (EPI) or confocal microscopy can be used to identify and quantify bacteria in the sample. In addition, bright field microscopy can be used to identify the type of type of bacteria in the sample (rods or cocci). The number of fluorescent light sources and bright field light sources can vary depending on the expected contents of a sample to be analyzed.

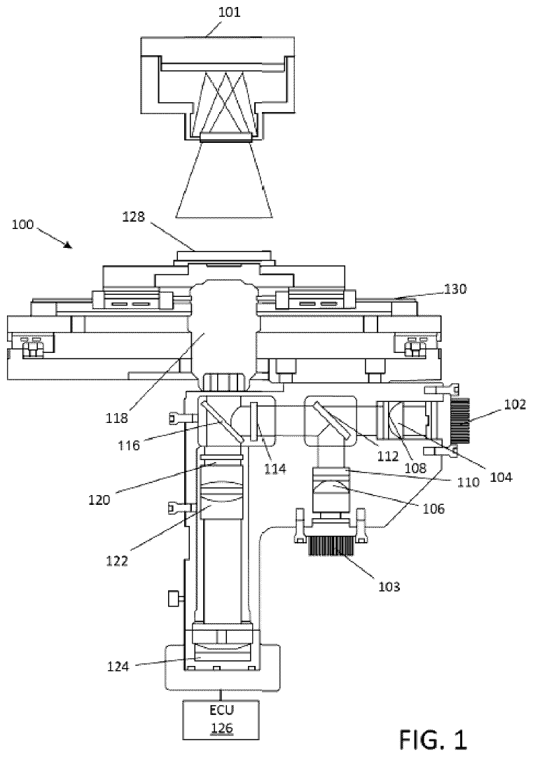

An example microscope apparatus of the disclosure is shown in FIG. 1. The apparatus 100 includes a digital microscope that includes a bright field light source 101, a fluorescent blue light source 102, a fluorescent ultraviolet light source 103, collector lenses 104 and 106, a blue excitation filter 108, an ultraviolet excitation filter 110, an excitation dichroic 112, a field lens 114, an imaging dichroic 116, an objective lens 118, a triband filter 120, a tube lens 122, a camera 124, and an electronic control unit (ECU) 126. The objective lens 118 may facilitate a bright field light source (not shown). A sample container 128 is shown positioned on X-Y stage 130.

In operation, the sample container 128 is placed along the path optical path of the microscope and illuminated with the bright field light source 101, the fluorescent blue light source 102 and/or the fluorescent ultraviolet light source 103. Sequentially or simultaneously, one of the fluorescent blue light source 102 or the fluorescent ultraviolet light source 103 illuminates the sample through dichroic 116, and the bright field light source can be applied in conjunction with or separately from the fluorescent light sources The sample interacts with the incident light and then generates a fluorescent signal that moves back down through the dichroic 116 to the camera 124. Alternatively or in addition, the bright field source is transmitted through the sample and objective lens 118 to the optics to the camera 124. The image(s) captured by the camera 124 are transmitted to the ECU 126 for automated analysis, as disclosed herein.

For scanning the sample, a Z-axis field of view (FOV) or multi-FOVs can be selected based on the amount of bacteria to be evaluated. Scanning of the container can include scanning the depth of the container (or an increment or increments thereof) at a single X-Y coordinate within the container/sample, scanning a single Z-axis depth at multiple X-Y coordinates of the container/sample, or scanning multiple Z-axis depths at multiple X-Y coordinates within the container/sample.

A Z-stack scan can cover a particular X-Y coordinate in a sample from the container bottom to the top in order to access the entire bulk fluid. Alternatively, a Z-stack scan can cover one or more increments of the Z-stack. Each increment of Z-axis depth may be set 1 to 50 μm based on the optics depth of field. For example, each increment may be 1 μm, 2 μm, 3 μm, 4 μm, 5 μm, or 6 μm, 10 μm, 20 μm, 30 μm, 40 μm or 50 μm, or within the range of 1 to 6μ, 2 to 5 μm, or 3-4 μm. Image acquisition can be either the X-Y motion first to cover all FOVs at a particular Z-axis depth, then follow Z-stack scanning or vice versa.

In various embodiments of the disclosure, the Z-stack scan covers between about 10% to about 100% of the Z-axis depth of the sample. For example, the Z-stack scan can cover 10%, 20%, 30%, 40%, 50%, 60%, 70%, 80%, 90% or 100% of the Z-axis depth of the sample. When the Z-stack scan covers less than 100% of the Z-axis depth of the sample, the scan can include contiguous or non-contiguous increments of the Z-stack. The scan may include the bottom of the sample, the top of the sample, neither, or both. In one embodiment, the scan includes contiguous increments accounting for about 25% to 75%, for example about 25%, 30%, 35%, 40%, 45% or 50%, 55%, 60%, 65%, 70% and 75% of the Z-axis depth without including the top or bottom of the sample. Z-axis depth can be selected such that it is at least above the point where the focus depth of field has immediate noise from settled white blood cells or other fluorescing cells, for example about 25-50 μm above the bottom of the sample container, for example 25 μm, 30 μm, 35 μm, 35 μm, 40 μm, 45 μm or 50 μm, above the bottom of the sample container. In samples with settled cells or other settled sample components that generate a large fluorescent signal, the Z-axis scan can be even further from the bottom of the container, or up to about 75 μm, e.g., 55 μm, 60 μm, 65 μm, 70 μm, or 75 μm, from the bottom of the container in order to avoid noise from settled components.

In various embodiments, less than the entire volume of the sample is scanned due to variations in sample settling. As discussed above, sample components settle at different rates, with smaller components such as bacteria, remaining in suspension the longest. And some sample components may float. Therefore, after a predetermined period of time the sample can be scanned to avoid the top and bottom of the sample to avoid scanning increments containing non-bacteria sample components. Depending on the height of the sample container, the increments may be at least 25 μm, 50 μm, 75 μm, 100 μm or more off the bottom of the sample to avoid interference from sample components at or near the bottom of the sample container. In addition, some sample components may float (e.g., cells). Therefore, scanning the top of the sample can be avoided.

As an example embodiment, images obtained 20-50 μm from the bottom of the container may be high enough depending on the depth of focus of the camera system. Similarly, the FOV can jump, for example, to 50, 75, or 100 microns depth and use those fixed depth locations for the analysis. Data generated from the full-scan experiments can be used to choose appropriate depths and settling times and build a correlation function from the abbreviated data set to determine the type, presence or amount of bacteria in a sample.

Within the X-Y plane, one or more X-Y coordinates within the sample can be scanned at one or more Z-depths. The size of a coordinate location area that can be scanned at a particular increment of the Z-stack is determined by the size of the focus of the microscope. For example, X-Y area in a field of view may be in the range 1 to 30 megapixels, which reflects an area of about 0.1 to about 3.0 mm². In an example sample cartridge having an X-Y plane of about 0.5 to about 1.0 cm², the number of focus spots that can be imaged in the plane with no or minimal overlap would be about 275-550.

Because bacteria can be expected to be uniformly distributed with the scanned sample, only a portion of the sample must be scanned in order to determine the type, presence and or amount of bacteria in the sample. In some embodiments, the total volume of the sample that may be scanned may be between about 1% to about 25%, for example about 10%. For quantitation of bacteria in the sample when less than 100% of the sample is scanned, the amount of bacteria in the portions of the sample that are scanned can be correlated to the amount of bacteria in the entire sample. In some embodiments 1 to about 100 FOVs are analyzed, for example, 1, 2, 3, 4, 5, 6, 7, 8, 9, 10, 20, 30, 40, 50, 60, 70, 80, 90 of 100 FOVs. In some embodiments, the number of FOVs that are analyzed are less than 50, less than 40, less than 30, less than 20, less than 10, or less than 5 FOVs.

The camera exposure time can vary in view of excitation light intensity, camera dynamic range and elapsed assay time. In some embodiments, camera exposure time is 50 ms, 100 ms, or 150 ms. Because the dye will intercalate into the bacteria over time, fluorescence emission from the stained bacteria will increase over time. Therefore, camera exposure time may be shorter near the end of a scan compared to the beginning. In some embodiments, the camera exposure time decreases over the course of scanning a sample at several X-Y and/or Z-axis depths by at least 25%, 50%, or 75%. Also, increased sample temperature can impact the stain uptake. Increasing exposure time will increase the signal when it is low, and decreasing exposure time will help pull the signal down if it is saturating the sensor.

At a given stain concentration and temperature, along with the susceptibility of the bacteria to allow the stain to uptake, then the fluorescent signal will increase with time as the stain moves towards equilibrium (and potentially starts to decrease if the light source continues after the stain is used and the stain begins to photo-bleach). Imaging the sample after a predetermined amount of time allows for component settling and stain uptake. Therefore one aspect of the disclosure optimizes time to result even when stain and fluorescent concentration has not reached equilibrium before sample scanning begins. According, in one embodiment, the system may use multiple exposure times that allow the fluorescent response to increase over the scan time which avoiding saturated signals. In other words, the system may adjust exposure times to be longer when fluorescent signal is low (at the beginning of predetermined amount of time), and shorter when fluorescent signal is higher (near the end of the predetermined amount of time). In various embodiments of the disclosure the exposure time can range from about 50 milliseconds (ms) to about 100 ms, or about 20 ms to about 140 ms. Example exposure times during a scan of a sample may be one or more of 20 ms, 30 ms, 40 ms, 50 ms, 60 ms, 70 ms, 80 ms, 90 ms, 100 ms, 110 ms and 120 ms, which may take into account camera response time, aperture size, and variations in fluorescent signal over time as noted above.

Changing X-Y coordinates of the Z-stack FOV in the sample can be accomplished by traditional automated microscope stages use lead screws with motors for precise positioning of X-Y coordinates. Z-stack images can be changed by leadscrew and motor or also use features like a liquid lens that can change focal position by applying an electric field to the liquid lens for extremely fine positioning. It is possible to move the sample container, the camera, or both. Traditionally, moving the cartridge and fixing the optics supports stability and protects against vibrational blur if the optics were to move.

### Methods for High Precision Bacterial Counting

Methods of the disclosure include detecting the type, presence or amount of bacteria in a biological sample. The methods include processing a native sample to suspend the sample in a liquid suspension medium by mixing the sample with surfactants and pH adjusting agents as described herein. Once the bacteria are suspended into a homogeneous liquid sample, bacteria in the sample can be stained with a fluorescent stain corresponding to bacteria. Bacteria stains can be selected as described herein such that the stain provides a sufficient signal to noise ratio and provides minimum optical background interference to avoid false positive bacteria identification. In some embodiments, all of the non-sample ingredients of the suspension liquid are in solid form and mixed with a diluent (e.g., water or buffer) and the native sample to provide the liquid sample.

After the bacteria staining, the sample may be added to a sample container having Z-axis height that allows for settling of components of the biological sample and allowing the components in the sample to settle for a predetermined amount of time. Following a predetermined amount of time, an image of the sample is obtained at a location comprising a unique set or sets of X-axis and Y-axis coordinates within a FOV of an increment of the Z-axis height (Z-stack image). The presence or absence bacteria within the FOV is determined and may be correlated to the type, presence or amount of bacteria in the sample container and/or native sample. In another embodiment, a plurality of FOVs can be imaged at the same or different set of X-Y coordinates within the sample.

Sample container Z-axis depth will determine when all non-bacteria components have settled. Normal cells (such as red or white blood cells) usually settle at a rate of around 1 μm per second. Therefore, in various embodiments, a predetermined amount of time will allow the non-dissolving sample components to settle at least enough such that the FOVs imaged by the apparatus of the disclosure will have minimal or no sample components other than bacteria as all or most other components will have settled to the bottom of the container or at least below the range of FOVs imaged according to the disclosure. In a clinical laboratory or point-of-care setting, an analysis time of less than ten minutes is helpful for delivery of time and labor savings. A set of Z-stack images (capturing successive images vertically through the bulk of a liquid sample) can be captured in a camera attached to the microscope. In some embodiments, scanning can begin before non-bacteria settling is complete by capturing a set of Z-stack images (capturing successive images vertically through the bulk of a liquid sample). The images can be compared by histogram analysis, for example, to separate the bacteria from the non-bacteria. For example, intensity values from scans of different Z-stack increments of the liquid sample intensity may be plotted as a histogram with the x-axis being depth in the chamber. The histograms may be used to develop algorithms to classify/quantify bacteria. In addition, a time series of histograms may be added to incorporate the settling profiles of sample components.

Under a fluorescence microscope, the stained bacteria can be seen with an appropriate optical fluorescence filter set based on the different excitation and emission wavelengths associated with the bacteria stains. Image analysis would have minimum optical background interference to avoid false positive bacteria identification since most other cells and objects in the sample will settle more rapidly than bacteria. As the unique property of bacteria, Z-dimension dispersion in a bulk fluid, a full or partial three dimensional Z-stack image rendering can be obtained for further analysis. In addition, optical image capture approaches can be applied to provide high image quality for better bacteria evaluation accuracy. These are high pixel resolution cameras with higher quantum efficiency and high-speed light shutters as well as confocal scanning or spinning-disk setting.

An image viewer program can adjust the contrast of the image to an optimal intensity allowing better visualization of fluorescence-stained bacteria. Additional brightfield image acquisition may be used to analyze and screen out non-specific fluorescence staining objects such as cell debris. The unique bacteria Z-dimension dispersion can be evaluated. The dispersion gradient is based on the container depth, the type of bacteria (rod or cocci), and the bacteria amounts. The gradient dispersion will be used to count the bacteria and differentiate the type of bacteria.

Zooming in on bright-field images may allow for determining a type of bacteria, for example distinguishing rods from cocci. Cocci tend to be one or a few pixels and will have a roughly circular shape. Rods will have an elongated shape (naturally bigger than cocci) and are identified from cocci based on that elongation. Once bacteria are identified in the fluorescent images, brightfield images of those cells can help to determine the morphology and separation of rod from cocci.

In some embodiment the FOV collected for a Z-stack image is above the settled elements at the bottom of the cartridge. When the bottom of the cartridge is sufficiently out of focus (invisible), reliable Z-stack images can be obtained. In another embodiment, the entire bulk sample can be scanned to obtain estimate of total bacteria load.

In various embodiments, an initial scan can be performed immediately once the cartridge is loaded and then repeat scans can be performed at set time intervals to compare differences that can provide inferences about the bacteria supporting separation from non-bacteria and typing using traditional or machine learning algorithm techniques.

If there are non-bacteria in a FOV, then those areas can be masked-off from the X-Y coordinates based on the size of the areas. Bacteria will be small—about one micron across or rods that are about one micron by two microns. Once an image is masked to only include bacteria-sized elements, metrics can be extracted from the remaining fluorescence images of one or more planes above the bottom of the container. In an aspect, integration of distinct fluorescence spots reflecting a presence of bacteria in a plane can provide a measure of bacteria load in the sample. Optionally, increasing complexity, such as integrating the fluorescent intensity values, can be used. The approach above avoids manual labeling of bacteria for algorithm training and counting of individual bacteria. Instead, the method uses a metric that is a monotonic function of bacteria load.

### Data Processing

By capturing brightfield and fluorescent images at each location, the method of the disclosure increases the accuracy of the identifying features of the bacteria. The fluorescent signal can be reduced to single parameter, such as magnitude of fluorescence intensity, or it can be used in using machine learning techniques to extract the needed information for classification. For example, machine learning approaches can evaluate images and not just mathematical extractions from images. Therefore, it is not necessary to use classical computer vision approaches to extract attributes from images that provide quantitative values that can then be used for classification. It is possible to use techniques that can include convolutional neural networks that can evaluate images that match reference images. Alternatively, machine learning techniques can use training data sets to indicate that bacteria is present in the sample and allow the machine learning logic to find the appropriate attributes as part of the training and then provide a metric for bacteria.

In some embodiments, an algorithm may mask off any fluorescence signal from a source that is too large to be bacteria and then quantifies the magnitude of fluorescence from the remaining image area, evaluating the dispersion of bacteria throughout the image (bacteria should be roughly uniformly distributed and nonuniformity would indicate something else, though it is possible for bacteria to cluster together and this would be a special case for the training algorithm). In another embodiment, an algorithm may consider bacteria size/shape within the image (e.g, by convolutional neural network or other) to determine what is and is not bacteria and then adding them up and comparing with a threshold or reporting quantity per FOV.

In some embodiments, an algorithm, program, or software may be used to quantify the amount of bacteria in a sample. In some embodiments, a computing device coupled to or in communication with the camera executes instructions (e.g., instructions stored in a memory of the computing device) in order to measure an amount of bacteria in the sample. Such instructions may be executed by a processor of the computing device in order to automate a portion of the measurement described above in relation to naked-eye techniques. For example, the instructions may be configured to measure the amount of bacteria in the sample by measuring or determining a number of pixels that correlates to the bacteria (e.g., corresponds to bacteria cells or an area of the image that consists of a bacteria cells) in an image of the sample. Additionally or alternatively, the instructions may cause the computing device to measure an amount of bacteria in the sample by measuring an area of an image of the sample that includes bacteria. Measuring the area that includes the bacteria may include determining a number of pixels of an image that relate to the bacteria. Differentiating the bacteria from the background of the image may include analyzing a numerical value associated with the pixels. For example, each pixel in an image could include a value corresponding to an amount of collected light, a level of intensity, brightness, coloration, greyscale, or another optical property of the pixel (e.g., a pixel in 8-bit image could be represented by a number between 0 and 255, where 0 corresponds to black and 255 corresponds to white). In a particular example, a threshold value could be set such that pixels with a value higher than the threshold are considered as comprising the bacteria, while those with a value lower than the threshold value are considered background. In such a case, determining a number of pixels that relate to the bacteria may include determining a number of pixels that are above or below some threshold value.

### Correlation Between Bacteria Counting Methods

As noted above, it is helpful for clinicians to be able to correlate results associated with traditional bacteria counting methods and high precision methods in order to facilitate diagnosis and treatment. Therefore, the disclosure provides methods for calibrating a bacteria detection method against traditional counting methods. In one embodiment, the method includes counting bacteria from a biological sample using traditional glass slide methods and/or colony counting methods. Sample bacteria counts from these methods can be compared to counts obtained using high precision methods.

Example method steps include counting bacteria on glass slides (with or without the use of immersion oil) and culturing bacteria from known serial quantities of bacteria from biological samples on culture plates. For example, a first plate sample, a second plate sample and so on can be obtained from serial diluted amount of a biological sample (e.g., ear cerumen collected from one or both ears of an animal). The known quantities may be determined, for example, by measuring optical densities of serially diluted samples as known in the art.

The diluted plate samples can then also be analyzed in a high precision method by using the sample from the plates having the predetermined quantities of bacteria and analyzing the samples in a high precision method that includes combining at least a portion of the samples with a fluorescent stain for bacteria to provide analysis samples. The analysis samples are introduced into sample containers having a Z-axis height that allows for settling of components of the analysis sample. Settling times can include, for example, about one to five minutes. Shorter times, e.g., three minutes, that allow for appropriate separation of sample components provide for shorter total analysis time.

Following the predetermined amount of time, the analysis samples can be analyzed by imaging a plurality of field of views (FOVs) that include increments of the Z-axis height in the analysis sample at a plurality of X-axis and Y-axis coordinates (X-Y coordinates) and determining a presence or amount of bacteria within each FOV. Scanning of FOVs can include increments of the Z-axis height off the bottom of the cartridge from about 10 μm to about 250 μm, for example 50 μm to 150 μm. About 10 to about 250 FOVs can be imaged, although fewer FOVs provide shorter overall analysis times. In an example, fewer than 50 FOVs are imaged. Imaging can include all increments with a selected range of a Z-axis heights or may include selected increments at one or more X-Y coordinates.

An example sample cartridge is in Vue Dx™ Ear Swab Cartridge the Example FOV and Z-axis heights are described above and can also include with the ear cartridge (e.g., in Vue Dx™ Ear Swab Cartridge) have a 3 minute incubation/settling time followed by 27 field of view images from 50 um through 150 um in increments of 25 um above the bottom of the cartridge and around focus points in the first 5 FOVs and 16th-20th FOVs.

The presence or amount of bacteria in the selected FOVs can be correlated to the number of bacteria in the analysis sample and the amounts of bacteria in the corresponding plate samples. In addition, amount obtained from the colony counting methods and the high precision methods can be compared to the amount of bacteria determined by a glass slide method.

In aspects of the disclosure, a transfer function is prepared to correlate the amount of bacteria in the samples obtained through the glass slide method, the colony counting methods and the high precision method. The quantity of the bacteria measured on the glass slide may be identified within one of a plurality of defined ranges of quantities of bacteria that can be correlated to clinical decision points associated with bacteria in a biological sample and resulting in a diagnosis of infection. See Table 1, above. Also, the quantities bacteria measured in the high precision method may also be identified as within one of the plurality of defined ranges of quantities of bacteria that are associated with the glass slide methods or the colony counting method.

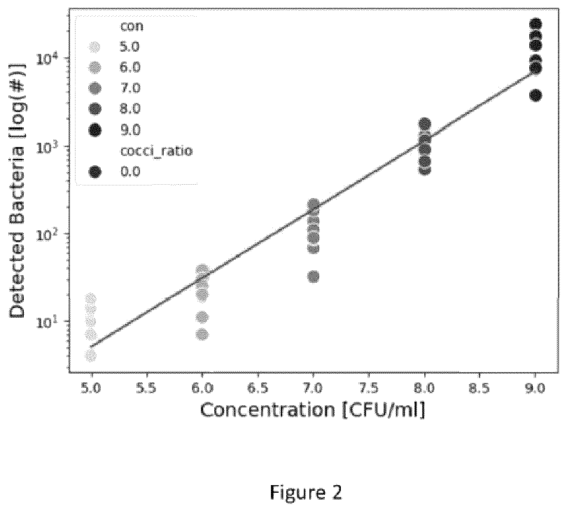

In one aspect, experimental data is used to train a neural network to quantify the bacteria load from an image looking at elevated locations in the Z-stack against the reference counts from quantitative plate cultures. The training uses, for example, the quantitative bacterial count as truth and the various high precision images to generate the logic. An example of how this training can occur from a titration of bacteria samples is shown in FIG. 2.

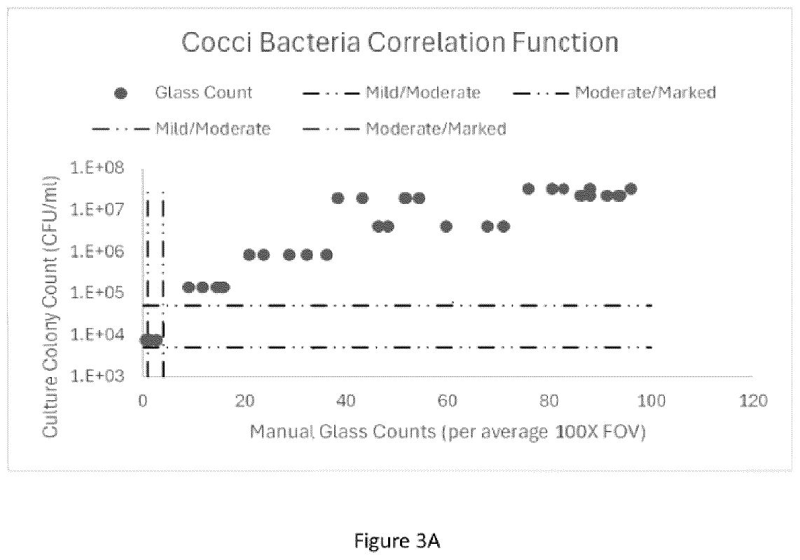

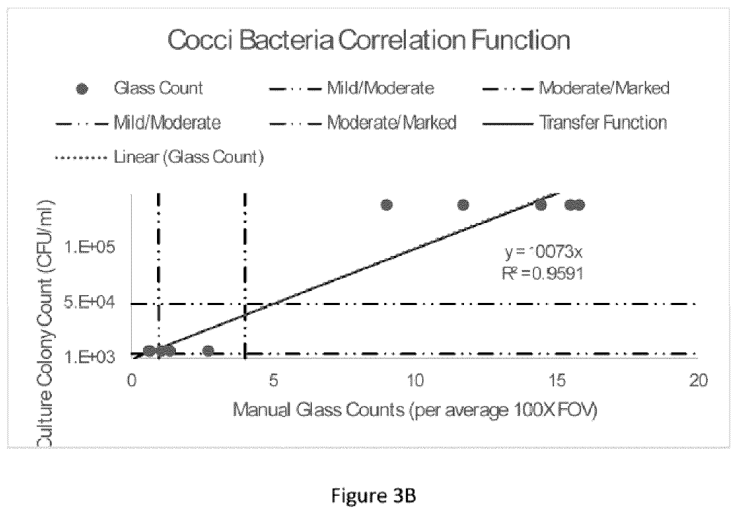

In an aspect, after the classifier is trained, a next experimental data set can be adopted. This set uses a titration of bacteria samples from the quantitative culture, high-precision measurement, and reference glass slide prep from a cotton swab. The quantitative culture helps to tie the amount of bacteria found on glass to the known performance of quantitation from the training approach. Once the data is complete, the algorithm output can be plotted on the y-axis, similar to FIG. 2, and the glass bacteria count can be plotted on the x-axis. Regression techniques can then be applied to the data to determine the transfer function that best represents the data including all inherent variation. A representation of that data can be shown in FIGS. 3A and 3B, which compares a glass slide manual review to quantitative culture counts as to find the number of CFU/ml by manually counting the culture plate colonies. Correlation is determined in the area near the clinical thresholds since the output will be reported in semiquantitative buckets (e.g. none, mild, moderate, and marked). It is possible to model the entire transfer function to provide quantitative results, but the semiquantitative approach is shown in FIGS. 3A and 3B. FIGS. 3A and 3B show a representative transfer function plot (3A) of a high-precision bacteria counting method compared to traditional glass reference method and zoomed in on the clinically relevant decision area (3B).

Once the transfer function is known it can be applied to the thresholds for clinical significance on a glass preparation from Table 1 to get the associated clinical thresholds for the high-precision measurement.

In one example, the high precision analysis device may train a machine learning model using data associated images of biological samples that share a characteristic with captured images of biological samples. The machine learning model may be trained using training data that shares a characteristic with a biological sample to be analyzed by the imaging device. Training the machine learning model may include inputting one or more training images into the machine learning model, predicting, by the machine learning model, an outcome of a determined condition of the one or more training images, comparing the at least one outcome to the characteristic of the one or more training images, and adjusting, based on the comparison, the machine learning model.

In some examples, the training data may include supervised learning, semi-supervised learning, or unsupervised learning. In some examples, training may include reinforcement learning.

The machine learning model may include an artificial neural network, a support vector machine, a regression tree, an ensemble of regression trees, or some other machine learning model architecture or combination of architectures.

In some examples, the machine learning model of the high precision device may be adjusted based on training such that if the outcome of a determined condition matches the characteristic of the training images, the machine learning model is reinforced and if the outcome of a determined condition does not match the characteristic of the training images, the machine learning model is modified. In some examples, modifying the machine learning model includes increasing or decreasing a weight of a factor within the neural network of the machine learning model. In other examples, modifying the machine learning model includes adding or subtracting rules during the training of the machine learning model.

### EXAMPLES

The Examples that follow are illustrative of specific embodiments of the disclosure.

They are set forth for explanatory purposes only, and are not intended to limit the scope of the disclosure.

### Example 1

A sample containing cocci strain Staphylococcus aureus was analyzed according to the method of the disclosure. The about 10⁷/mL bacteria sample was harvested from an agar plate and suspended in about 1 ml of a suspension liquid containing 7.9 g/L of sodium chloride, 1.65 g/L of Tris-HCl, 0.8 g/L of EDTA and 2.35 g/L of Tris-base. SYTO-13™ dye was used and prepared at the final concentration of 2.5 μM to stain the sample.

Stained sample was loaded on an Incyto™ cell counter slide as a sample container. A Nikon Ti microscope with a confocal scan head was used to capture the Z-stack images in 5 μm fields of view (FOV).

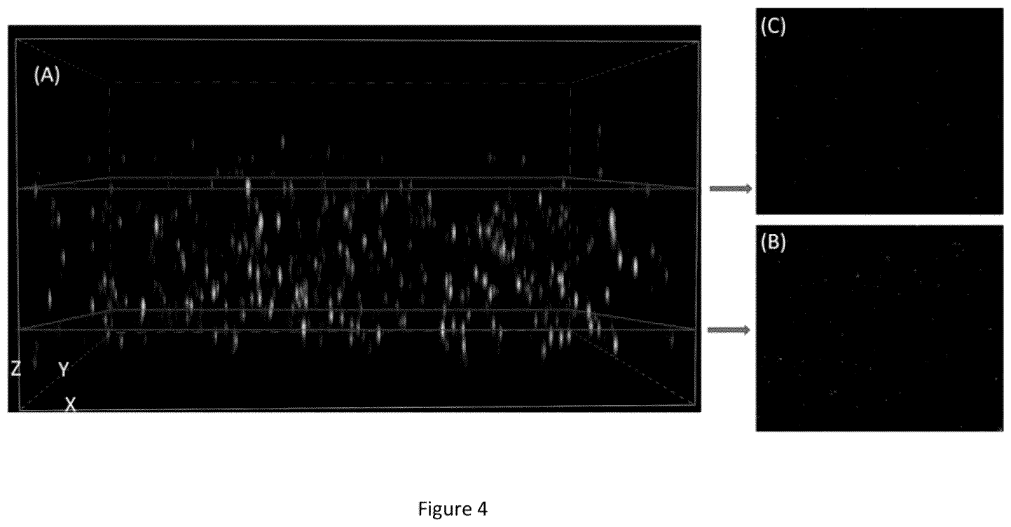

FIG. 4 is a gray-scale reproduction of a Z-stack image having a depth of 100 μm (insert A) and a Z-stack dispersion of 60 μm of Z-stack increments of 5 μm within the 100 μm depth. Each object represents one coccus, which in the original image is shown as a green fluorescent signal. Insert B of FIG. 4 shows a plane of Z-stack images close to the container bottom. Insert C of FIG. 4 shows a plane of Z-stack images close to the cell counter top. Inserts B and C show a concentration difference suggesting a gradient dispersion that is a function of settling time and can provide insight regarding the bacteria based on its time-based vertical concentration histograms.

### Example 2

An experiment to compare the difference in Z-dispersion for both cocci and rod was conducted using the same conditions as those used in Example 1, except the bacteria was Serratia marcescens, which are rods.

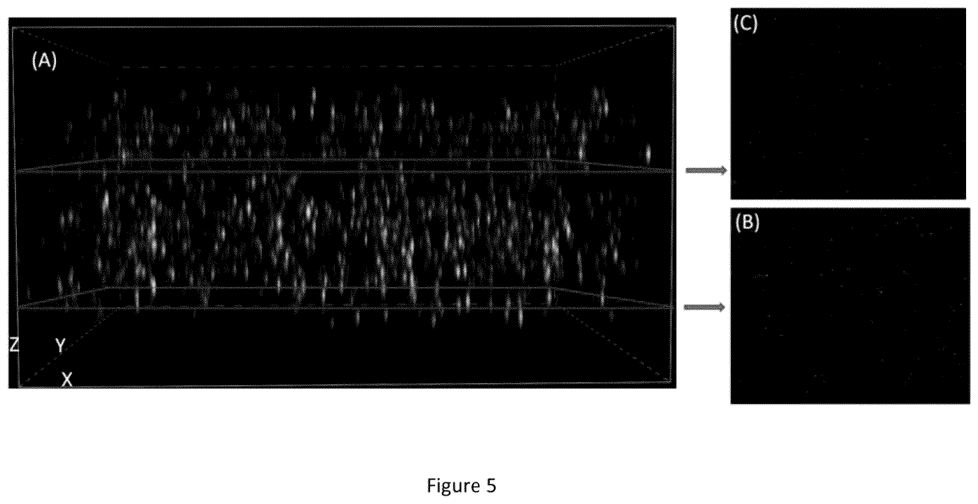

FIG. 5 show a 3-D dispersion in a sample having a Z-stack depth of 100 μm and the Z-dispersion is about 70 μm. Each object in Insert A represents one rod. Insert B shows a plane of Z-stack images close to the cell counter bottom. Insert C shows a plane of Z-stacks image close to the cell counter top. Each fluorescence-stained rod can be seen as an individual population.

Both cocci and rod have different Z-dispersion properties and could be used to differentiate the type of bacteria. While not particularly shown in FIGS. 4 and 5, enlarged brightfield images can show distinguishable images of rods and cocci. Rods will have an elongated shape (naturally bigger than cocci) and are identified from cocci based on that elongation.

### Example 3

To test the unique bacteria Z-dispersion, an E. coli containing liquid sample was prepared to a concentration at about 10⁷/mL bacteria in deionized water and compared to a liquid sample containing dust with similar size and concentration. Bacteria and dust samples were filled in separate Incyto™ cell counter slides as sample containers. Sample imaging was performed on a Nikon Ci microscope with a brightfield image acquisition process. The Z-scanning range with brightfield imaging was set to include the top and bottom of a container. Z-step images of 5 μm each were collected and rendered into a 3-D volume view.

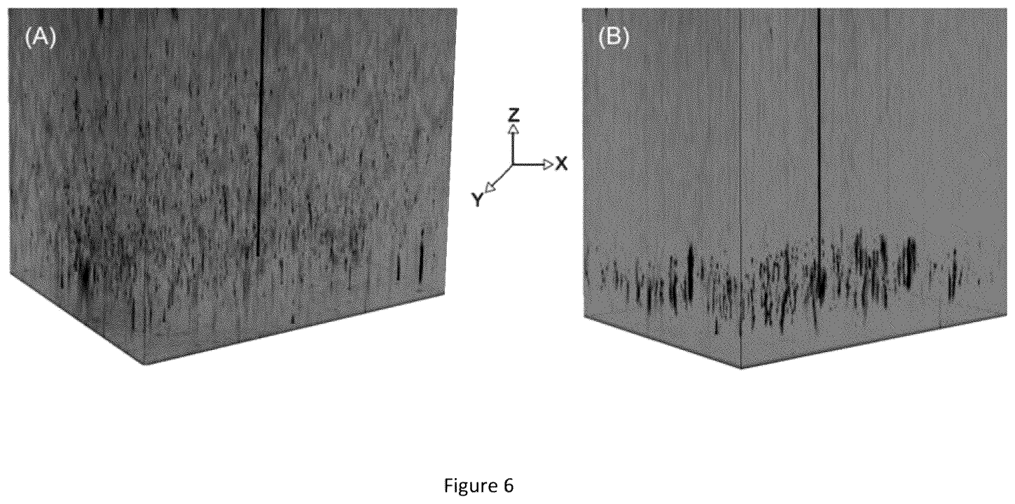

FIG. 6 shows a composite image of the entire sample container sample with either (A) E. coli, or (B) dust, after a predetermined amount of time of twenty minutes. Each black colored object represents a bacteria or dust population. The dust settled close to the cell counter bottom after the predetermined amount of time.

### Example 4: Preparation of Bacterial Samples Collected from Agar

A culture of cocci strain Staphylococcus pseudointermedius was aseptically prepared with a disposable inoculating loop (BD, Ref 220215) on Tryptic Soy Agar with 5% Sheep's Blood plates (Northeast Laboratory Services, Part Number P1100) using quadrant streaking for isolation. Plates were incubated inverted plate for 18-24 hours at 37±1° Celsius. Following incubation, varying target concentrations of Staphylococcus pseudointermedius were prepared using filtered autoclaved deionized water and a ThermoScientific GENESYS™ 30 visible spectrophotometer programmed to a fixed wavelength of 600 nm and zeroed using filtered autoclaved deionized water (see Table 2). The varying concentrations were obtained by transferring colonies of bacteria to a conical tube containing filtered autoclaved deionized water and homogenizing using a vortex.

One mL of bacterial suspension was added to a disposable cuvette, and the optical density was measured. Optical density was increased with additional bacterial colonies from the agar plate, or decreased with filtered autoclaved deionized water as needed, until the target optical density was obtained. Table 2 reflects the optical density measurements for each target bacterial concentration.

TABLE 2

| | Target Concentration (in CFU/mL) | | | |
|---|---|---|---|---|
| | 10² | 10³ | 10⁴ | 10⁵ |
| | CFU/mL | CFU/mL | CFU/mL | CFU/mL |
| Target OD600 | 0.375 | 0.897 | 1.495 | 2.212 |
| Dilutions and Plating | Undiluted | Undiluted | 10⁻¹ | 10⁻³ |
| (n = 2 each | | 10⁻¹ | 10⁻² | |
| concentration) | | | | |

Each target concentration was tested (n=5) to evaluate consistency of the method. Three sets of data were collected for each:
(1) microscopic counts on a glass microscope slide at 100× with immersion oil (Example 5);
(2) concentration of bacteria rolled off of a standard cotton swab onto a glass microscope slide (Example 5);
(3) concentration of bacteria transferred when tested using the in Vue Dx™ (IDEXX Laboratories, Inc.) method for ear swab testing (Example 6).

### Example 5: Determination of Bacteria By Glass Slide Methods

Target concentrations of bacterial samples shown in Table 1 were analyzed for bacterial load with a glass slide procedures (1) using immersion oil and (2) rolled off of a cotton swab. In both methods, glass slides are prepped by rolling the bacteria off of the swab onto them. A first slide is evaluated microscopically at 100× with immersion oil. The second slide is used to obtain the concentration of bacteria coming off of the swab onto the glass by using a second polyester swab to collect the contents back from the slide, and using dilutions and plating to obtain colony counts. As an example, determination of 10 bacteria/100× HPF in method (1), reflects that 1000 CFU/mL came off of the swab.

For both counting methods, five glass microscope slides were marked on the underside with a solvent-resistant marker with a 3 mm line to serve as a guide, to ensure each swab is rolled approximately the same distance along the slides. A 70% isopropyl alcohol prep wipe was used to sterilize the top of the slide and allowed to dry. Slides were numbered 1-5 on the frosted edge.

The slides were allowed to air dry overnight and stained using Hemaspray Romanowsky stain in an Aerospray automated stainer. 20 fields of the glass slide were reviewed at 100× with immersion oil and cocci counts were recorded for each.

### Example 6: Correlation of Bacteria on Glass Slide and Colony Counting

Bacterial samples collected from agar plates as in Example 4 (Table 2) were analyzed by counting colony forming units on the agar plates.

The bacterial suspension in the 5 mL tube was diluted down to 1:1000. Three culture plates for each dilution (undiluted, 1:10, 1:100, and 1:1000) were prepared by plating 0.1 mL of each using spread plating technique with sterile L-shaped cell spreaders (Celltreat, Product Code 229617). Plates were incubated inverted plate for 18-24 hours at 37±1° C. Colonies were counted, and bacterial concentration was calculated using the standard formula: CFU/mL=(Average Plate Count×Dilution Factor)/Volume Plated (mL).

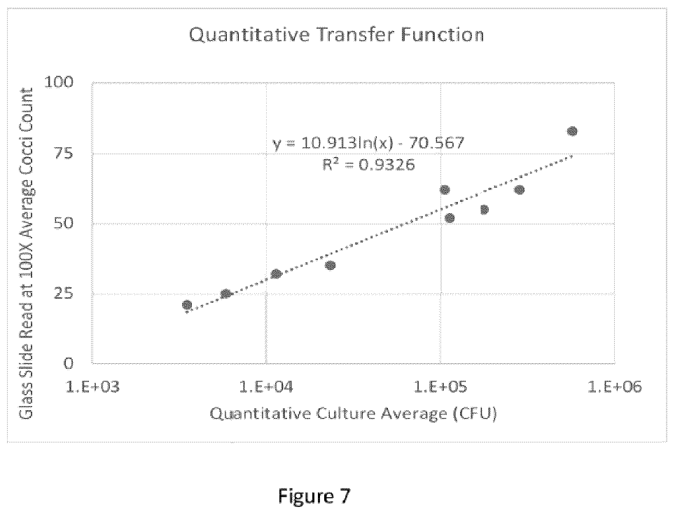

Colony counts were compared to the amount of bacteria determined by the glass slide method (100× with immersion oil). FIG. 7 shows a transfer function comparing the bacteria determined by the two methods. The data shows a logarithmic relationship between colony forming units and glass slide counts, and therefore is shown as a semi-log plot resulting in the best fit logarithmic function presenting as a straight line.

### Example 7: Determination of Bacteria with High Precision

Samples analyzed in Example 4 were also analyzed by the high precision method as described herein. A Puritan sterile polyester tipped applicator (swab) that was used to collect the contents transferred to the slide in Example 5 was placed into a conical tube containing 5 mL filtered autoclaved deionized water. The well-mixed bacterial suspension was divided into five, 100 uL aliquots, labeled 1-5. Using Puritan sterile cotton tipped applicator (swab), the contents of 1 aliquot were collected, and the swab was inserted into an in Vue Dx™ ear swab diluent vial (see U.S. provisional application Ser. No. 63/604,544, which is incorporated by reference herein) containing 500 uL diluent (water, Tris-HCl, EDTA, Trizma® base, Sodium Chloride, Dioctyl sulfosuccinate (AOT), proclin 300 (biocide). The swab was swirled while pushing against the internal ribs and withdrawn while squeezing the vial to extract any excess diluent taken up by the swab. An in Vue Dx™ lyophilized stain cap was inserted into the diluent vial, and the vial was mixed by inversion 5 times. The top of the dropper cap was removed, and all contents of the vial were added to one side of the in Vue Dx™ Ear Swab Cartridge (see U.S. provisional application Ser. No. 63/615,571, which is incorporated by reference herein). The prepared cartridge was then inserted into the instrument and a run was initiated.

The analyzer provides a settling time that also supports intercalation of the fluorescent stain into the cells. The analyzer then also finds the best focus position of the settled plane aided by focusing beads that are part of the lyophilized reagent. After finding the settled plane, the focus is shifted into the fluid bulk where images are taken above the settled plane.

Scans with the ear cartridge have a 3 minute incubation/settling time followed by 27 field of view images from 50 um through 150 um in increments of 25 um above the bottom of the cartridge and around focus points in the first 5 FOV and 16th-20th FOV.

### Example 8: Preparation of Transfer Function from Algorithm Assessment of Bacterial Load by High Precision Method to Quantitative Culture Colony Forming Unit Method

The algorithm can be trained to provide a quantitative value corresponding to the bacteria concentration found in the quantitative culture by including images above the settled plane so that the vast majority of non-bacteria fluorescing cells have settled and will not be part of the image. White blood cells may be included in the sample so that any far out of focus noise from those cells will be trained out of the calculation. As an alternative, fluorescing pixels in the bulk may be counted and the area those pixels can integrated to provide that as a quantitative measure of bacterial load in that plane.

The WBC's are prepared by adding 2 mL of whole blood in EDTA to 20 mL of 1× lysing solution. The mixture is inverted 5-10 times, allowed to rest for 5 minutes, and centrifuged at 1050 rpms for 10 minutes. The supernatant is decanted off, and the WBC pellet is resuspended in 20mL of 1% BSA 1×PBS to wash. The centrifugation step is repeated and the supernatant is decanted and the pellet is resuspend in 1 mL of 1% BSA 1×PBS. The sample is then run on ProCyte Dx® analyzer (IDEXX Laboratories, Inc) to determine WBC concentration. Once the desired WBC concentration is achieved, the isolated WBC mixture is added to the bacterial suspension in the diluent vial prior to the addition of the lyophilized stain cap and instrument run.

A machine learning model can used to determine bacterial load as a function of an image of the bacteria in a plane above the surface of the cartridge as described in Example 7 to the determination of bacterial load as describe in Example 4. The data can demonstrate that the bacterial CFU follows a predictable function when compared with quantitative culture measured by high precision. The data in FIGS. 3 and 3B show a representative output of an AI model of bacterial load against quantitative culture.

### Example 9: Integrated Transfer Function and Threshold Determination from a Glass Slide Count to an AI Model Determination Of Bacterial Load

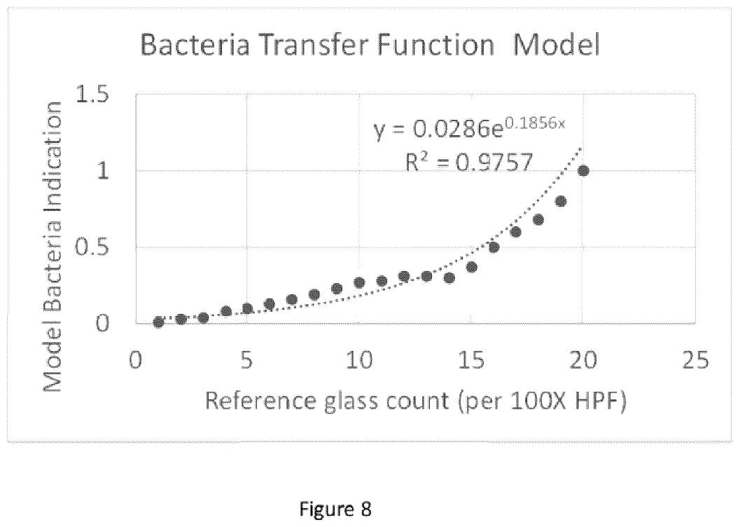

The data from FIGS. 5 and 6 can then be integrated to generate the desired transfer function from glass slide bacterial count to AI model bacterial load value, as shown in FIG. 7. The threshold for positive infection can then be taken from guidance to review bacterial load in ears on a glass slide as represented in FIG. 8.

The singular forms of the articles “a,” “an,” and “the” include plural references unless the context clearly indicates otherwise. For example, the term “a compound” or “at least one compound” can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

What is claimed is:

1. A method for detecting a type, presence or amount of bacteria in a liquid comprising:
staining a liquid sample with a fluorescent stain for bacteria,
introducing the stained liquid sample into a sample container having a Z-axis height that allows for settling of components of the liquid sample;
allowing the components in the liquid sample to settle for a predetermined amount of time;
imaging a Z-axis field of view (FOV) comprising an increment of the Z-axis height in the liquid sample at a location comprising an X-axis and Y-axis coordinate (X-Y coordinate); and
determining the presence or absence of bacteria within the FOV and correlating the type, presence or amount of bacteria in the FOV to the type, presence or amount of bacteria in the liquid sample.

2. The method of claim 1, wherein an image of the FOV is obtained with a confocal microscope or an epifluorescence (EPI) microscope.

3. The method of claim 1, wherein the imaging comprises obtaining a plurality of FOVs at the X-Y coordinate and correlating the type, presence or amount of bacteria in the plurality of FOVs to the type, presence or amount of bacteria in the liquid sample.

4. The method of claim 1, wherein the Z-axis height is between about 0.1 to 1 mm.

5. The method of claim 4, wherein a depth of field of the increment of the Z-axis height is about 1 μm to 10 μm.

6. The method of claim 1, wherein determining the presence or absence of bacteria within the FOV comprises comparing an image of the FOV to a predetermined image indicative of the presence or type of bacteria in the FOV.

7. The method of claim 1, wherein the imaging comprises determining a fluorescence spectra of stained bacteria in the FOV.

8. The method of claim 1, wherein the imaging comprises determining a fluorescence spectra of stained bacteria in the FOV and determining a strain of the bacteria in the FOV based upon the fluorescence spectra.

9. The method of claim 1, further comprising processing a native human, animal, environmental or industrial sample to obtain the liquid sample by mixing the native sample with a reagent comprising a surfactant and a buffer.

10. The method of claim 1, wherein the sample is ear cerumen, peripheral blood, a solid tumor, fine needle aspirate, urine or body cavity fluid.

11. The method of claim 1, further comprising obtaining a bright-field image of the FOV.

12. The method of claim 1 wherein the determining comprises analyzing an image with a computing device coupled to or in communication with a camera that images a FOV and executes instructions stored in a memory of the computing device.

13. A method for calibrating a bacterial detection method, comprising
(a) counting bacteria from a biological sample using a glass slide method to obtain a reference count,
(b) culturing bacteria from a portion of the biological sample on a first plate;
(c) counting the bacteria on the first plate;
(d) collecting a first plate sample from the first plate having a first predetermined quantity of bacteria;
(e) analyzing the first plate sample in a high precision analysis method for determining an amount of bacteria in the first plate sample,
(f) correlating the amount of bacteria determined in step (e) to an amount of bacteria in the plate sample determined in step (c), and
(g) comparing the amount of bacteria in the first plate sample to the amount of bacteria in the reference count.

14. The method of claim 13, wherein the high precision analysis method comprises:
(i) combining at least a portion of the first plate sample with a fluorescent stain for bacteria to provide an analysis sample,
(ii) introducing the analysis sample into a sample container having a Z-axis height that allows for settling of components of the analysis sample;
(iii) allowing components in the analysis sample to settle for a predetermined amount of time;
(iv) imaging a Z-axis field of view (FOV) comprising an increment of the Z-axis height in the liquid sample at a location comprising plurality of X-axis and Y-axis coordinates (X-Y coordinates); and
(v) determining the amount of bacteria within the FOV.

15. The method of claim 13, further comprising repeating steps (d) through (g) with a second plate sample comprising a second predetermined quantity of bacteria collected from the first plate or a second plate comprising as second bacteria culture comprising bacteria from the biological sample cultured on a second plate.

16. The method of claim 13, wherein the first plate sample is diluted prior to step (e).

17. The method of claim 13, wherein the first predetermined quantity of bacteria is determined by measuring the optical density of the first diluted plate sample.

18. The method of claim 13, further comprising collecting at least a portion of the first bacteria culture with a swab or a loop, optionally further comprising collecting at least a portion of the second bacteria culture with a swab or a loop.

19. The method of claim 13, further comprising preparing a transfer function to correlate the amount of the bacteria determined in step (e) to the amount of bacteria in the reference count.

20. The method of claim 13, wherein the amount of the bacteria measured on the glass slide in step (a) is identified within one of a plurality of defined ranges of quantities of bacteria.

21. The method of claim 20, wherein the defined ranges are correlated to clinical decision points associated with bacteria in a biological sample and resulting in a diagnosis of infection.

22. The method of claim 13, wherein the quantity of bacteria measured in step (e) is identified as within one of a plurality of defined ranges of quantities of bacteria.

23. The method of claim 13, wherein the counting the bacteria on the plate comprises counting colony forming units on the plate.
